(19) United States
(12) Patent Application Publication
(10) Pub. No.: US 2009/0151394 A1
(43) Pub. Date: Jun. 18, 2009

(54) JEWELRY EXTRACTION AND INSERTION INSTRUMENT AND KIT

(76) Inventor: Harry J. Ake, Mullica Hill, NJ (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/958,863

(22) Filed: Dec. 18, 2007

(51) Int. Cl. A44C 7/00; A61B 17/34

(52) U.S. Cl. 63/12; 606/188

(57) ABSTRACT

A disposable, threaded, multi-component body jewelry instrument provides safe and reliable extraction and insertion of body jewelry and stretching of a piercing site. Body jewelry can be inserted and extracted from either end of the piercing site. A piercing site can be stretched from either end of the piercing site. Body jewelry with female and male attachable ends can be accommodated. In an example configuration, the multi-component body jewelry instrument comprises a first component having a female threaded portion, a second component having dual male threaded portions, and a third component having a female threaded portion. Another example embodiment comprises a kit containing the multi-component body jewelry instrument.

### TECHNICAL FIELD

The technical field generally relates to jewelry. More specifically, the technical field relates to an instrument for extracting and inserting body jewelry, and to a kit comprising a jewelry extraction and insertion instrument.

### BACKGROUND

It is not uncommon for persons wearing body jewelry to desire to change jewelry. Changing body jewelry requires extracting jewelry from a piercing site and re-inserting jewelry at the piercing site. Also, it is not uncommon for body jewelry to accidentally come out of the piercing site, thus requiring re-insertion of the body jewelry. Additionally, wearers of body jewelry are known to intentionally stretch the piercing site. Stretching the piercing site requires removing the body jewelry from the piercing site, stretching the piercing site, and re-inserting jewelry into the stretched piercing site.

Extraction and insertion of body jewelry can be tedious and difficult. Typically, a wearer travels to a professional piercing establishment to have the jewelry extraction and/or insertion procedure performed. Often, a charge is associated with the procedure. The procedure typically involves prodding and physical manipulation of the piercing site. Often, insertion is not accomplished on the first attempt. It is difficult to safely and routinely probe a piercing site from either direction of the piercing site. Commonly, for example when working with nose jewelry, the connection between components of body jewelry and the insertion instrument is lost, requiring more prodding and probing. Not surprisingly, the procedure can amount to an uncomfortable experience for the wearer.

Maintenance of body piercing instruments can be burdensome for the piercing professional. Existing body jewelry instruments are expensive and require sterilization. Sterilization usually requires an autoclave, which can be expensive to purchase and maintain.

### SUMMARY

A multi-component body jewelry instrument provides safe and reliable extraction and insertion of body jewelry from either end of a piercing site. The multi-component body jewelry instrument also facilitates stretching of a piercing site from either end of the piercing site. In an example configuration, the multi-component body jewelry instrument is disposable. The components of the multi-component body jewelry instrument can be coupled via various means, such as threaded means, pressure fit means, or the like.

In an example embodiment, the multi-component body jewelry instrument comprises a first component having a handle and a female threaded portion, a second component having dual male threaded portions, and a third component having a female threaded portion and a blunt, tapered tip. The first component is configured to threadably attach to the second component via one of the second component's male threaded portions. The third component is configured to threadably attach to the second component via the other one of the second component's male threaded portions. The third component is configured as a solid, tapered probe or pin. In example embodiments, the multi-component body jewelry instrument is configured with various gauge dimensions compatible with the body jewelry being inserted/extracted such that a coupling between body jewelry and the body jewelry instrument is maintained until the insertion, extraction, and/or stretching procedure is completed. In alternate example embodiments, components of the multi-component body jewelry instrument are attached via non-threaded attachment means, such as snap-on connections, pressure fit connections, or the like. In alternate example embodiments, the multi-component body jewelry instrument can be made of various materials, such as metals, glasses, ceramics, and/or plastics. In alternate example embodiments, the multi-component body jewelry instrument can be a disposable sterilized item made of appropriate materials, such as plastics, for example.

In another example embodiment, the multi-component body jewelry instrument is provided as a kit for consumer use. The kit can comprise various elements such as a single use sterilized disposable multi-component body jewelry instrument, a pair of exam gloves, sterile lubrication jell, sterile plastic haemostatic forceps, aftercare products, and an instruction pamphlet or booklet providing illustrations and instructions for using the multi-component body jewelry instrument, aftercare suggestions, helpful hints for healing the piercing site, or the like.

### BRIEF DESCRIPTION OF THE DRAWINGS

The foregoing and other objects, aspects and advantages of a multi-component body jewelry instrument will be better understood from the following detailed description with reference to the drawings.

FIG. 1 is an illustration of an example multi-component body jewelry instrument.

FIG. 2 is a more detailed illustration of the third component 18.

FIG. 3 is a flow diagram of an example process for utilizing the multi-component body jewelry instrument.

FIG. 4 is an illustration of an example body jewelry known as a Labret stud.

FIG. 5 is an illustration of a nose stud and a component of the multi-component body jewelry instrument.

FIG. 6 is an illustration of an example kit comprising a multi-component body jewelry instrument.

### DETAILED DESCRIPTION OF ILLUSTRATIVE EMBODIMENTS

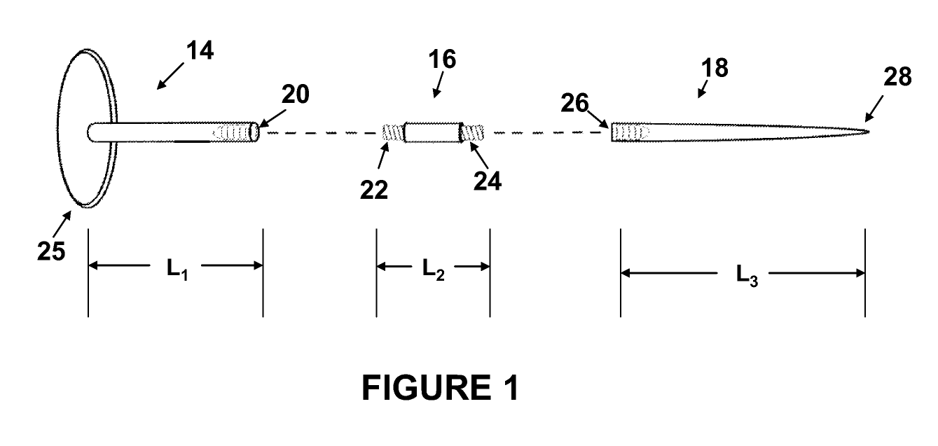

FIG. 1 is an illustration of a multi-component body jewelry instrument 12 comprising a first portion 14, and second portion 16, and a third portion 18. The first portion 14 comprises a proximate end 25 and a distal end 20. The second component comprises a proximate end 22 and a distal end 24. The third component comprises a proximate end 26 and a distal end 28.

In an example configuration, the proximate end 25 of the first component 14 comprises means for grasping and manipulating the multi-component body jewelry instrument 12. The proximate end 25 can comprise any appropriate means for grasping and manipulating the multi-component body jewelry instrument 12, such as a handle or the like. As depicted in example FIG. 1, the proximate end 25 comprises a handle in the form of a tab, similar to a key for a wind-up clock. The distal end 20 comprises an attachable portion configured to be attachable to the second component 16. The distal end 20 comprises means for coupling to the proximate end 22 of the second component 16. The coupling means of the distal end 20 can comprise any appropriate means for coupling to the second component 16, such as threaded means, pressure fit means, snap fit means, or a combination thereof. As depicted in example FIG. 1, the distal end 20 comprises a female threaded portion.

The second component 16 comprises a proximate end 22 and a distal end 24. The proximate end 22 comprises an attachable portion configured to be attachable to the distal end 20 of the first portion 14. The distal end 24 comprises an attachable portion configured to be attachable to the proximate end 26 of the third portion 18. The attachable portion of the proximate end 22 can comprise any appropriate means for coupling the proximate end 22 of the second component 16 to the distal end 20 of the first component 14, such as threaded means, pressure fit means, snap fit means, or a combination thereof. As depicted in example FIG. 1, the proximate end 22 comprises a male threaded portion. The attachable portion of the distal end 24 can comprise any appropriate means for coupling the distal end 24 of the second component 16 to the proximate end 26 of the third component 18, such as threaded means, pressure fit means, snap fit means, or a combination thereof. As depicted in example FIG. 1, the distal end 24 comprises a male threaded portion.

The third component 18 comprises a proximate end 26 and a distal end 28. The proximate end 26 comprises an attachable portion configured to be attachable to the distal end 24 of the second component 16. The proximate end 26 comprises means for coupling the proximate end 26 of the third component 18 to the distal end 24 of the second component 16. The coupling means of the proximate end 26 can comprise any appropriate means for coupling to the second component 16, such as threaded means, pressure fit means, snap fit means, or a combination thereof. As depicted in example FIG. 1, the proximate end 26 comprises a female threaded portion. The distal end 28 of the third component 18 comprises a tapered portion having an outer diameter less than the outer diameter of the proximate end 26.

In example configurations, the lengths of the components 14, 16, and 18 can vary in accordance with expected use. For example, if the multi-component body jewelry instrument is to be used in conjunction with nose jewelry, the component 18 may be shorter than if the multi-component body jewelry instrument is to be used in conjunction with other body parts (e.g., belly button, ear, eyebrow). For example, the length L₁ of the first portion 14 can be between 1 inch and 2 inches; the length L₂ of the second portion can be between ½ inch to 1½ inches; and the length L₃ of the third portion can be between 1 inch and 3 inches. It is to be understood that the above enumerated lengths are exemplary and not limited thereto.

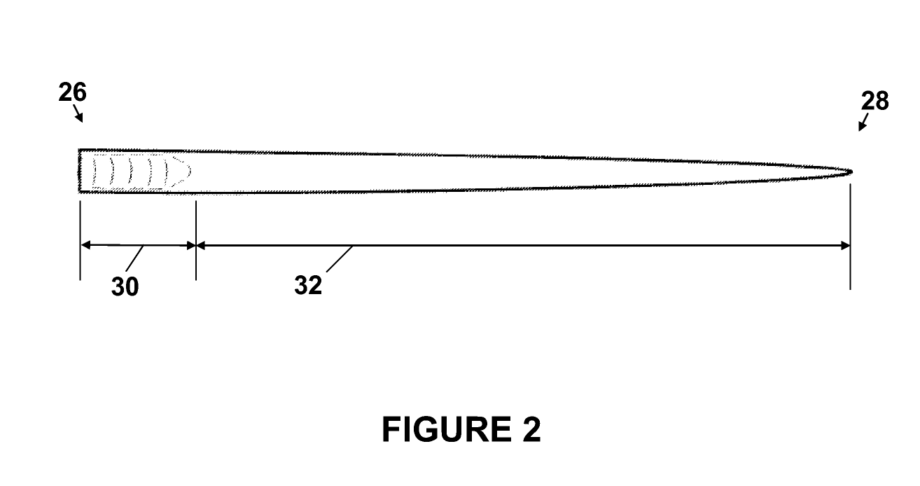

FIG. 2 is a more detailed illustration of the third component 18. In an example configuration, the third component 18 comprises a tapered distal end 28 having a smaller outer diameter than the outer diameter of the proximate end 26. As depicted in example FIG. 2, the third component 18 comprises a first portion 30 and a second portion 32. The first portion 30 extends from the proximate end 26 to the second portion 32. The second portion 32 extends from the first portion 30 to the tip of the distal end 28. In an example embodiment, the tip comprises a non-cutting, blunt edge. The outer diameter of the first portion 30 is essentially constant. That is, the outer diameter of the first portion is constant within manufacturing limits of the multi-component body jewelry instrument 12 and its components. The outer diameter of the first portion 30 can be of various sizes depending upon the size of the body jewelry to be inserted/extracted with the multi-component body jewelry instrument. In example embodiments, the outer diameter of the first portion 30 can be any of the diameters shown in Table 1 below. It is to be understood that the outer diameters shown in Table 1 are exemplary and are limited thereto. Further, it is to be understood that the outer diameter may vary within appropriate manufacturing tolerance.

TABLE 1
Nominal Outer Diameter

| Gauge | mm | inches |
|---|---|---|
| 10 | 2.5857 | 0.1018 |
| 11 | 2.3038 | 0.0907 |
| 12 | 2.0523 | 0..0808 |
| 13 | 1.8263 | 0.0709 |
| 14 | 1.6256 | 0.0640 |
| 15 | 1.4478 | 0.0570 |
| 16 | 1.2903 | 0.0508 |
| 17 | 1.1481 | 0.0452 |
| 18 | 1.0326 | 0.0403 |

The outer diameter of the second portion 32 is monotonically increasing from the distal end 28 to the first portion 30. The second portion 32 is solid. The second portion 32 is non-cannular. The monotonically increasing outer diameter of the second portion 32 is utilizable to stretch a piercing site. It is emphasized that the second portion 32 is not a hypodermic needle. In contrast to a hypodermic needle, the second portion 32 is solid, non-cannular, comprises a blunt tip at the distal end 28, and comprises no inner hollow portion.

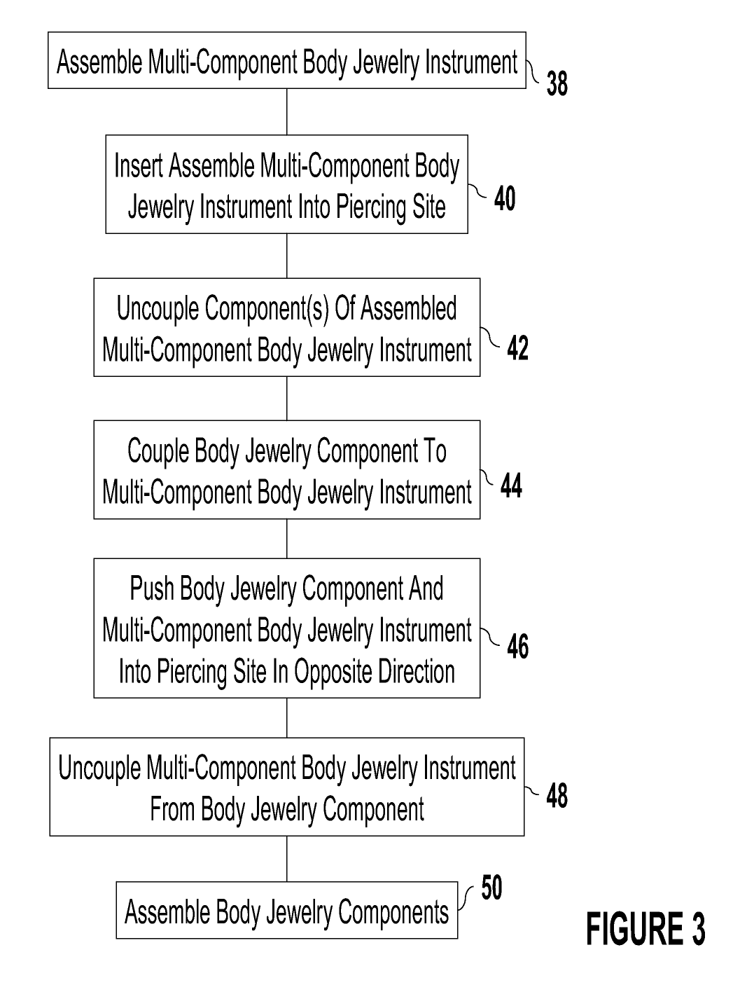

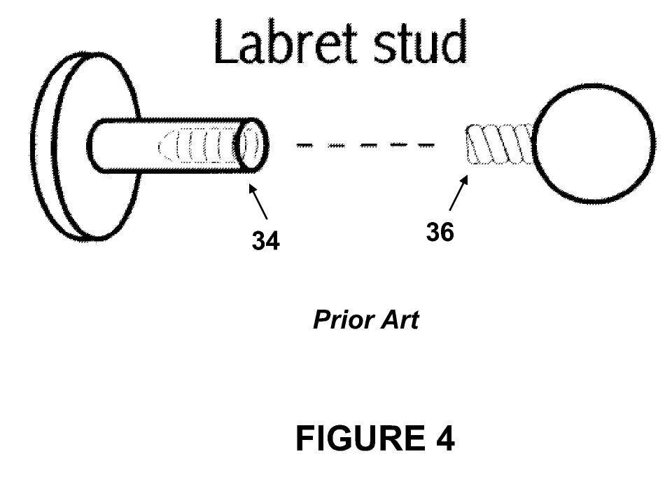

FIG. 3 is a flow diagram of an example process for utilizing the multi-component body jewelry instrument. FIG. 1 and FIG. 4 will be referenced to describe the process depicted in FIG. 3. FIG. 4 is an illustration of an example body jewelry known as a Labret stud. Referring now to FIG. 3, to utilize the multi-component body jewelry instrument 12, at step 38, the multi-component body jewelry instrument 12 is assembled prior to insertion into a piercing site. Assembling the multi-component body jewelry instrument 12 comprises coupling the distal end 20 to the proximate end 22 and coupling the distal end 24 to the proximate end 26. The handle of the proximate end 25 can be used to manipulate and maneuver the assembled multi-component body jewelry instrument 12. The tapered tip of the distal end 28 of the assembled multi-component body jewelry instrument 12 is inserted into and through the piercing site at step 40. The assembled multi-component body jewelry instrument 12 can be inserted from any end or in any direction into the piercing site. The multi-component body jewelry instrument 12 is inserted into the piercing site until the appropriate portion of the multi-component body jewelry instrument 12 has been pushed through the piercing site. In an example embodiment, the multi-component body jewelry instrument 12 is inserted through the piercing site until a desired portion of the first component 14 passes through the piercing site. Once the desired portion of the multi-component body jewelry instrument is inserted through the piercing site, the multi-component body jewelry instrument can be disassembled to allow the body jewelry to be attached thereto.

At step 42, the multi-component body jewelry instrument is partially disassembled. And, at step 44, a portion of the body jewelry is coupled to the multi-component body jewelry instrument 12. At this point, either a male end or a female end of body jewelry can be coupled to the multi-component body jewelry instrument 12. For example, if the portion of the body jewelry comprises a female end, such as female end 34 of the Labret stud depicted in FIG. 4, the third component 18 of the multi-component body jewelry instrument 12 is uncoupled from the second portion 16 and the female end 34 of the body jewelry is coupled to the male end 24 of the second component 16 at step 44. If the portion of the body jewelry comprises a male end, the third component 18 and the second component 16 are uncoupled from the multi-component body jewelry instrument 12. At step 44, the male end 36 of the Labret stud is coupled to the female end 20. At step 46, the component or components coupled to the body jewelry component are pushed or pulled back into the piercing site. When the portion where the body jewelry component is coupled to the multi-component body jewelry instrument is pushed through the piercing site, the multi-component body jewelry instrument is uncoupled from the body jewelry component at step 48. The remaining component of the body jewelry is coupled to the other component of the body jewelry at step 50.

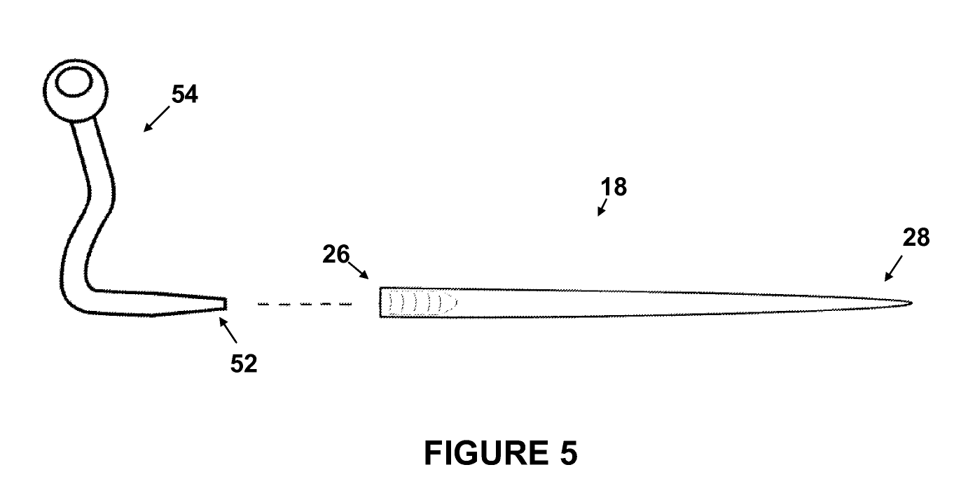

In an example embodiment, the multi-component body jewelry instrument is configured to receive a non-threaded piece of body jewelry. As depicted in FIG. 5, a piece of body jewelry 54 comprises a nose stud. The nose stud comprises a tapered end 52. The female end 26 can be threaded, tapered without threads, or a combination thereof. In an example embodiment, the multi-component body jewelry instrument is made of plastic or other appropriate resilient material, such that the insertion of the tapered end 52 into the female end 26 forms a snug pressure fit. Thus, the nose stud 54 coupled to the component 18 can be utilized to insert and/or extract the nose stud 54. In an example configuration, the component 18 can be manufactured to comprise a non-threaded female end 26 having dimensions that allow for a snug fit of the tapered end 52 of the body jewelry 54. As depicted in FIG. 5, the body jewelry 54 is a nose stud. It is to be understood however, that the body jewelry 54 can comprises any type of body jewelry having a tapered end.

In use, the body jewelry 54 can be inserted through a piercing site on a nose from the inner side of the nasal cavity or from the outside of the nasal cavity. In an example configuration the component 18 can be manufactured to be longer when expected to be used in conjunction with nose jewelry than when expected to be used for other body parts.

To utilize the multi-component body jewelry instrument with the nose stud 54, the tapered end 52 of the nose stud 54 is inserted into the female end 26 of the component 18 of the multi-component body jewelry instrument 12, and the blunt tip at the distal end 28 of the component 18 is inserted into the piercing from the exterior of the nasal cavity wall. The component 18, with the body jewelry 54 attached, is pulled and/or pushed through the piercing hole until the tapered end 52 and a sufficient portion of the body jewelry 54 are within the nasal cavity. At this point the component 18 is disconnected from the body jewelry 54, and the body jewelry 54 is properly installed.

Additionally, the multi-component body jewelry instrument 12 can be used to locate and open a piercing hole, and insert body jewelry, from the inside of the nasal cavity wall. To accomplish this, the handle on the proximate end 25 can be used to manipulate and guide the assembled multi-component body jewelry instrument 12 (see FIG. 1). The blunt tip of the distal end 28 of the assembled multi-component body jewelry instrument 12 is used to locate and penetrate the piercing hole from the inside of the nasal cavity wall. The multi-component body jewelry instrument 12 is inserted through the piercing site until the distal end 20 of the first component 14 extends therethrough. The multi-component body jewelry instrument 12 is then disassembled while the first component 14 remains in the piercing site with the distal end 20 extending therethrough. At this point the tapered tip 52 of the body jewelry 54 is inserted into the distal end 20 of the first component 14. The first component 14 is pulled back into the nasal cavity with the body jewelry 54 attached thereto. This results in the tapered end 52 of the body jewelry 54 being pulled into the nasal cavity. When a sufficient portion of the body jewelry 54 is pulled into the nasal cavity, the distal end 20 of the first component 14 can be disconnected from the tapered end 52 of the body jewelry 54. At this point, the body jewelry 54 is properly installed.

Each component of the multi-component body jewelry instrument can be comprise any appropriate material, such as, for example, gold, silver, surgical steel, titanium, glass, plastic, wood, horn, amber, stone, bamboo, silicone, ivory, bone, zircon, bronze, niobium, or any combination thereof. In an example configuration, the multi-component body jewelry instrument is configured to be disposable. A disposable multi-component body jewelry instrument can comprise a less expensive material such as plastic or the like.

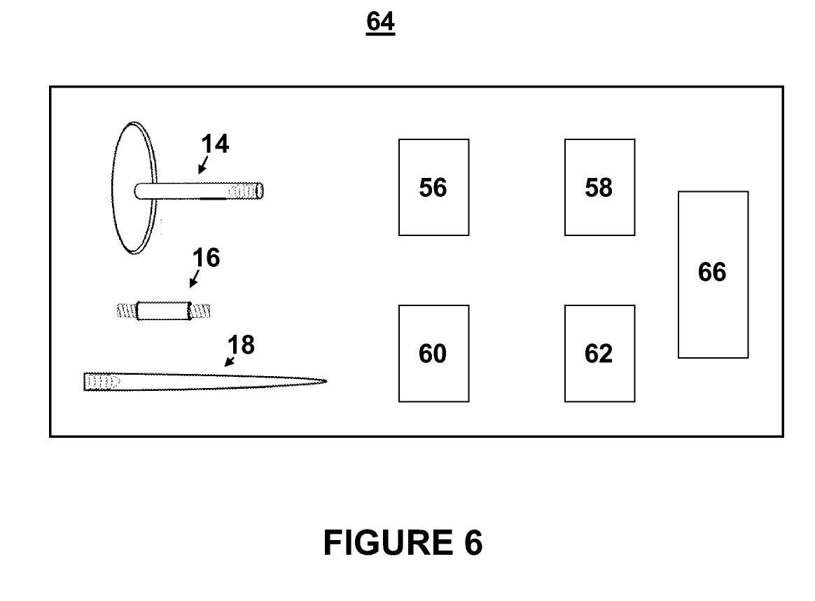

FIG. 6 is an illustration of an example kit 64 comprising a multi-component body jewelry instrument. In an example embodiment, the kit 64 is configured for consumer use. The kit 64 can comprise various elements that can be used in association with the multi-component body jewelry instrument. In an example embodiment, the kit comprises the first component 14 of the multi-component body jewelry instrument, the second component 16 of the multi-component body jewelry instrument, the third component of the multi-component body jewelry instrument, a pair of exam gloves 56, sterile lubrication jell 58, sterile plastic haemostatic forceps 60, and an instruction pamphlet or booklet 62 providing illustrations and instructions for using the multi-component body jewelry instrument, aftercare suggestions, helpful hints for healing the piercing site, after-care products 66 such as medicated soap, lotions, sea salt preparations, or the like. The kit 64 can comprise any combination of the components shown in FIG. 6.

The multi-component body jewelry instrument can be configured in various gauges, lengths, and shapes. For example, although the multi-component body jewelry instrument is depicted as being straight in Figures herein, the multi-component body jewelry instrument can be curved. The attachable portions of the multi-component body jewelry instrument can be configured in various gauges to accommodate various configurations of body jewelry. The components of the multi-component body jewelry instrument can be configured in various lengths. The components of the multi-component jewelry instrument can be configured to be threadably attached, pressure fit attachable, or a combination thereof. For example, the first component 14 and the second component 16 can be configured to be threadably attached and the second component 16 and the third component can be configured to be pressure fit attachable. As another example, the first component 14 and the second component 16 can be configured to be pressure fit attachable and the second component 16 and the third component can be configured to be threadably attached. The sizes of the components of the multi-component body jewelry instrument can vary. For example the proximate end 22 and the distal end 24 of the second component 16 can be of different sizes. For example, the proximate end 22 can have a greater outer diameter than the outer diameter of the distal end 24 of the second component 16. Alternatively, the proximate end 22 can have a lesser outer diameter than the outer diameter of the distal end 24 of the second component 16. The multi-component body jewelry instrument can be utilized with any type of body jewelry, such as ear jewelry, nose jewelry, eye brow jewelry, lip jewelry, tongue jewelry, or bell button jewelry, for example.

While a multi-component body jewelry instrument has been described in connection with the various embodiments of the various figures, it is to be understood that other similar embodiments can be used or modifications and additions can be made to the described embodiment for performing the same function of a multi-component body jewelry instrument without deviating therefrom. Therefore, a multi-component body jewelry instrument should not be limited to any single embodiment, but rather should be construed in breadth and scope in accordance with the appended claims.

What is claimed is:

1. A multi-component body jewelry instrument comprising:
a first component comprising:
a proximate end comprising a handle; and
a distal end comprising an attachable portion configured to be attachable to a second component of the multi-component body jewelry instrument;
the second component comprising:
a proximate end comprising an attachable portion configured to be attachable to the first component; and
a distal end comprising an attachable portion configured to be attachable to a third component of the multi-component body jewelry instrument; and
a third component comprising:
a proximate end comprising an attachable portion configured to be attachable to the second component of the multi-component body jewelry instrument; and
a distal end comprising a blunt tip.

2. The instrument in accordance with claim 1, wherein:
the third component comprises a non-cannular tapered distal portion;
a tip of the non-cannular tapered distal portion is the blunt tip of the third component;
the non-cannular tapered distal portion comprises a monotonically increasing outer diameter extending from the tip.

3. The instrument in accordance with claim 1, wherein:
the attachable portion of the distal end of the first component comprises a female threaded portion configured to be compatible with the proximate end of the second component;
the attachable portion of the proximate end of the second component comprises a male threaded portion configured to be compatible with the distal end of the first component;
the attachable portion of the distal end of the second component comprises a male threaded portion configured to be compatible with the proximate end of the third component; and
the attachable portion of the proximate end of the third component comprises a female threaded portion configured to be compatible with the distal end of the second component.

4. The instrument in accordance with claim 1, wherein:
the attachable portion of the distal end of the first component comprises a pressure fit attachable portion configured to be compatible with the proximate end of the second component;
the attachable portion of the proximate end of the second component comprises a pressure fit attachable portion configured to be compatible with the distal end of the first component;
the attachable portion of the distal end of the second component comprises a pressure fit attachable portion configured to be compatible with the proximate end of the third component; and
the attachable portion of the proximate end of the third component comprises a pressure fit attachable portion configured to be compatible with the distal end of the second component.

5. The instrument in accordance with claim 1, wherein:
the attachable portion of the distal end of the first component comprises a female threaded portion configured to be compatible with the proximate end of the second component;
the attachable portion of the proximate end of the second component comprises a male threaded portion configured to be compatible with the distal end of the first component;
the attachable portion of the distal end of the second component comprises a pressure fit attachable portion configured to be compatible with the proximate end of the third component; and
the attachable portion of the proximate end of the third component comprises a pressure fit attachable portion configured to be compatible with the distal end of the second component.

6. The instrument in accordance with claim 1, wherein
the attachable portion of the distal end of the first component comprises a pressure fit attachable portion configured to be compatible with the proximate end of the second component;
the attachable portion of the proximate end of the second component comprises a male pressure fit attachable configured to be compatible with the distal end of the first component;
the attachable portion of the distal end of the second component comprises a threaded portion configured to be compatible with the proximate end of the third component; and
the attachable portion of the proximate end of the third component comprises a threaded portion configured to be compatible with the distal end of the second component.

7. The instrument in accordance with claim 1, wherein the first component comprises a body jewelry component.

8. The instrument in accordance with claim 1, wherein at least one of the first component, the second component, or the third component comprises at least one of plastic, metal, gem stone, bone, horn, wood, ivory, ceramic, or glass.

9. The instrument in accordance with claim 1, wherein the instrument is configured to be disposable.

10. The instrument in accordance with claim 1, wherein an outer diameter of the proximate end of the second component differs from an outer diameter of the distal end of the second component.

11. A kit comprising:
a first component of a multi-component body jewelry instrument, the first component comprising:
a proximate end comprising a handle; and
a distal end comprising an attachable portion configured to be attachable to a second component of the multi-component body jewelry instrument;
the second component of the multi-component body jewelry instrument, the second component comprising:
a proximate end comprising an attachable portion configured to be attachable to the first component; and
a distal end comprising an attachable portion configured to be attachable to a third component of the multi-component body jewelry instrument; and
a third component of the multi-component body jewelry instrument, the third component comprising:
a proximate end comprising an attachable portion configured to be attachable to the second component of the multi-component body jewelry instrument; and
a non-cannular tapered distal end.

12. The kit in accordance with claim 11, further comprising at least one of:
exam gloves;
lubrication gel;
haemostatic forceps;
an aftercare product; and
instructions.

13. The kit in accordance with claim 11, wherein the first component, the second component, and the third component are configured to be disposable.

14. The kit in accordance with claim 11, wherein the first component, the second component, and the third component comprise plastic.

15. The kit in accordance with claim 11, wherein:
the third component comprises a non-cannular tapered distal portion;
a tip of the non-cannular tapered distal portion is the blunt tip of the third component;
the non-cannular tapered distal portion comprises a monotonically increasing outer diameter extending from the tip.

16. The kit in accordance with claim 11, wherein an outer diameter of the proximate end of the second component differs from an outer diameter of the distal end of the second component.

18. The kit in accordance with claim 11, wherein:
the attachable portion of the distal end of the first component comprises a female threaded portion configured to be compatible with the proximate end of the second component;
the attachable portion of the proximate end of the second component comprises a male threaded portion configured to be compatible with the distal end of the first component;
the attachable portion of the distal end of the second component comprises a male threaded portion configured to be compatible with the proximate end of the third component; and
the attachable portion of the proximate end of the third component comprises a female threaded portion configured to be compatible with the distal end of the second component.

19. The kit in accordance with claim 11, wherein:
the attachable portion of the distal end of the first component comprises a pressure fit attachable portion configured to be compatible with the proximate end of the second component;
the attachable portion of the proximate end of the second component comprises a pressure fit attachable portion configured to be compatible with the distal end of the first component;
the attachable portion of the distal end of the second component comprises a pressure fit attachable portion configured to be compatible with the proximate end of the third component; and
the attachable portion of the proximate end of the third component comprises a pressure fit attachable portion configured to be compatible with the distal end of the second component.

20. The kit in accordance with claim 11, further comprising body jewelry, wherein dimensions of the first component, the second component, and the third component are compatible with the body jewelry.
